(19) United States
(12) Patent Application Publication
(10) Pub. No.: US 2013/0281747 A1
(43) Pub. Date: Oct. 24, 2013

(54) COBALT PHOSPHINE ALKYL COMPLEXES FOR THE ASYMMETRIC HYDROGENATION OF ALKENES

(71) Applicant: The Trustees of Princeton University, Princeton, NJ (US)

(72) Inventors: Paul CHIRIK, Princeton, NJ (US); Jordan M. Hoyt, Palm Harbor, FL (US); Max R. Friedfeld, Priceton, NJ (US)

(21) Appl. No.: 13/838,835

(22) Filed: Mar. 15, 2013

Related U.S. Application Data
(60) Provisional application No. 61/613,321, filed on Mar. 20, 2012.

(51) Int. Cl. C07C 5/03

(52) U.S. Cl. CPC C07C 5/03
USPC 585/277; 556/12; 585/275

(57) ABSTRACT

Disclosed herein are manganese, iron, nickel, or cobalt compounds having a bidentate ligand and the use of these compounds for the hydrogenation of alkenes, particularly the asymmetric hydrogenation of prochiral olefins.

### CROSS REFERENCE TO RELATED APPLICATIONS

The present application claims the benefit of U.S. Provisional Application No. 61/613,321, filed on Mar. 20, 2012, which is herein incorporated by reference in its entirety.

### STATEMENT REGARDING FEDERALLY SPONSORED RESEARCH OR DEVELOPMENT

A part of this invention was made with government support under Grant # CHE 1026084 awarded by the National Science Foundation. The government has certain rights in this invention.

### FIELD OF THE INVENTION

This invention relates to transition metal-containing compounds, more specifically to manganese, iron, cobalt, or nickel complexes having bidentate ligands, and the use of these compounds to catalyze the hydrogenation of olefins, preferably prochiral olefins. The present invention also relates to methods of making these transition metal-containing compounds.

### BACKGROUND OF THE INVENTION

Transition metal catalyzed olefin hydrogenation is a fundamental reaction in chemical synthesis. One category within catalyzed olefin hydrogenation is asymmetric hydrogenation of prochiral olefins, which is important in producing enantiopure fragrances, agrochemicals, biofuels, and pharmaceutical compositions. Present technologies for these hydrogenation reactions rely on iridium, rhodium, platinum, and ruthenium-based catalysts, and these precious metals are expensive, toxic and have fluctuations in supply. An example of this reaction was reported by Bell et al. in Science 2006, 311, 642-644, where an iridium catalyst was shown to be effective for asymmetric hydrogenation of prochiral olefins. There is no disclosure in Bell et al. of first row transition metal complexes or that such complexes would useful for this reaction.

Based on the drawbacks of present technologies, outlined above, an attractive route for transition metal catalyzed olefin hydrogenation is the use of first-row transition metals such as manganese, iron, cobalt, and nickel in catalysts for these hydrogenation reactions. These transition metals are terrestrially abundant and inexpensive compared to the precious metals currently in use. Cobalt starting materials are especially cheaper than the precious metals currently used in most hydrogenation reactions, and Zhu, Janssen, and Budzelaar (Organometallics 2010, 29, 1897-1908) have shown that (py)₂COR₂ (py=pyridine; R=CH₂SiMe₃) is an excellent starting material for forming cobalt complexes. Further, a variety of bidentate phosphine ligands have been developed in chemical industry and are available in bulk quantities, and these ligands can form chemical bonds with cobalt to form diphosphine cobalt dialkyl compositions of matter. These two factors suggest that the disclosed compounds have the potential to reduce costs in commercial asymmetric hydrogenations by replacing current iridium, rhodium, and ruthenium catalysts.

First row transition metal complexes, especially those of iron, cobalt, and nickel, are known, and the ligands thereof include monophosphines, bidentate ligands coordinate through phosphine, nitrogen, and/or oxygen atoms, tripodal, tridentate phosphine ligands, and bis(amino)pyridine ligands coordinated to a transition metal through each nitrogen atom. Some of these complexes have been shown to catalyze the hydrogenation of olefins.

Examples of cobalt complexes having monophosphine ligands coordinated to the cobalt atom have been disclosed in Yamamoto et al. (Chem. Comm. 1967, 2, 79-80), Pu et al. (J. Am. Chem. Soc., 1968, 90(25), 7170-7171), Hidai et al. (Tetrahedron Lett. 1970, 20, 1715-1716), Klein et al. (Chem. Ber. 1976, 109, 1453-1464), Li et al. (Z. Anorg. Allg. Chem., 2005, 631, 3096-3099), and Wadepohl et al. (Organometallics 2005, 24, 2097-2105). These complexes share a common moiety of Co(PPh₃)₃, where three discreet triphenylphosphine molecules coordinate to the cobalt atom.

Yamamoto et al. disclosed isolation of the Co(PPh₃)₃ moiety by coordinating a nitrogen molecule to the cobalt atom and that complexes having this moiety are effective for oligermizing olefins (e.g. dimerizing and trimerizing ethylene and propylene). However, there is no disclosure or suggestion in this reference that these compounds could catalyze olefin hydrogenation. Pu et al. reported that complexes having the Co(PPh₃)₃ moiety are effective for hydrogenation of ethylene and other easily hydrogenated olefins, but these cobalt complexes are known to be unstable.

Li et al. disclosed the synthesis of cobalt complexes starting from [CoMe₃(PMe₃)₃]. Methane and PMe₃ are each liberated by the reaction of this starting complex with imines having a phenol moiety. There is no disclosure or suggestion in Li et al. that cobalt complexes would be useful for hydrogenating olefins. Klein et al. disclosed pentacoordinated d⁷-complexes of cobalt having a general formula of CoX₂L_{3 (X=Cl, Br, I, CH}₃; L=P(CH₃)₃). Several reactions with these complexes are disclosed in Klein et al., but hydrogenation of olefins is not included or suggested by this reference.

Wadepohl et al. disclosed coordination of olefin to a (H)Co(PPh₃)₃ compound, which shares the same moiety disclosed in Yamamoto et al. and Pu et al., and that this compound is effective for β-elimination of the hydride to the olefin. However, each of these references disclosed that the three triphenylphosphine ligands remain coordinated to the cobalt atom during these reactions, and therefore the reference does not disclose or suggest that complexes having bidentate phosphine ligands or two monophosphine ligands coordinated to a cobalt atom could catalyze olefin hydrogenation. Last, Hidai et al. disclosed the use of [CoH(CO)(PPh₃)₃] to catalyze the hydrogenation of cyclohexene, but this compound was active at high reaction temperatures and very high hydrogen pressures. Further, the presence of AlEt₃ increased conversion of cyclohexene. These factors suggest that the cobalt complexes of Hidai et al. are inefficient at hydrogenating cyclohexene.

Each of Hendrikse and Coenen (J. Catal. 1973, 30, 72-78) and Hendrikse et al. (Int. J. Chem. Kinet. 1975, 7, 557-574) reported kinetic studies for the hydrogenation of cyclohexene catalyzed by Co(PPh₃)₃-containing complexes. No other cobalt complexes were reported nor were any studies presented for the hydrogenation of prochiral olefins.

Examples of first row transition metal complexes having bidentate ligands are disclosed in Chow et al. (Inorg. Chim. Acta, 1975, 14, 121-125), Ohgo et al. (Bull. Chem. Soc. Jpn., 1981, 54, 2124-2135), Corma et al. (J. Organomet. Chem. 1992, 431, 233-246), Klein et al. (Eur. J. Inorg. Chem. 2003, 240-248), Nindakova et al. (Russ. J. Org. Chem. 2004, 40, 973), and Imamura et al. (Chem. Lett. 2006, 35(3), 260-261).

Chow et al. disclosed the synthesis of several four-coordinated cobalt complexes having bidentate phosphine ligands bound to the cobalt atom. However, there is no disclosure or suggestion in the cited reference of using these compounds or compounds having a similar structure for catalyzing the hydrogenation of olefins. The complexes of Klein et al. did not catalyze any reactions with olefins, the authors stating that no “catalytic transformation of the olefins was observed.” Imamura et al. does not disclose any hydrogenation reactions. Each of Ohgo et al., Nindakova et al., and Corma et al. disclosed hydrogenation reactions, but none of these references disclose the hydrogenation of simple alkenes with high conversion percentages.

Complexes of tridentate, tripodal phosphine ligands bound to cobalt through the three phosphine atoms have been shown to catalyze olefin hydrogenation reactions. DuBois and Meek disclosed the synthesis of cobalt complexes with these ligands in Inorg. Chem. 1976, 15(12), 3076-3082, and disclosed the hydrogenation of 1-octene catalyzed by complexes having similar structures in Inorg. Chim. Acta. 1976, 19, L29-L30. Additional complexes of cobalt and tripodal phosphine ligands were disclosed by Orlandini and Sacconi in Inorg. Chim. Acta. 1976, 19, 61-66 and by Rupp et al. in Eur. J. Inorg. Chem. 2000, 523-536. However, all of these cited references fail to disclose or suggest that complexes of cobalt and bidentate ligands could catalyze the hydrogenation of olefins.

Monfette et al. (J. Am. Chem. Soc. 2012, 134(10), 4561-4564) disclosed complexes having tris-chelating bis(amino)pyridine ligands bound to cobalt, and that these complexes are efficient for the asymmetric hydrogenation of olefins. Similarly, Sauer et al. (Inorg. Chem. 2012, 51, 12948-12958) disclosed tris-chelating 1,3-bis(2-pyridylimino)isoindolates compounds and cobalt complexes thereof which can hydrosilylate ketones in an enantioselective manner, but the catalyst were not efficient at this reaction. There is no disclosure or suggestion in these references of any first row transition metal complexes having bidentate ligands bound to the metal center. Further, there is no disclosure or suggestion in Sauer et al. of olefins or prochiral olefins, that such olefins are hydrogenated, or that the disclosed cobalt complexes of this reference are efficient at enantioselective hydrogenation of prochiral olefins.

Zhang et al. (Angew. Chem. Int. Ed. 2012, 51, 12102-12106) disclosed tridentate P—N—P pincer ligands (bis[2-(dicyclohexylphosphino)ethyl]amine) and cobalt complexes thereof. The metal source was (py)₂CoNs₂, and these complexes were reported to be efficient at hydrogenating C═C, C═O, and C═N bonds within a variety of compounds. Further, there is no disclosure or suggestion of any first row transition metal complexes having bidentate ligands bound to the metal center, and that these complexes are efficient for hydrogenation of unsaturated bonds.

Last, Niewahner and Meek (Inorg. Chim. Acta. 1982, 64, L123-125) disclosed the hydrogenation of olefins catalyzed by rhodium complexes having trichelating phosphine ligands. There is no disclosure or suggestion of using first row transition metals as catalysts for these reactions in this reference.

In view of the foregoing, the present inventors have sought to synthesize first row transition metal containing complexes and bidentate ligands that are efficient for hydrogenating olefins. The present inventors have found that the disclosed compounds are useful for this purpose. The compounds of the present invention also have utility for, inter alia, pharmaceutical companies that work on asymmetric hydrogenation in their pharmaceutical production.

### BRIEF SUMMARY OF THE INVENTION

One object of the present invention is to provide transition metal-containing compounds having bidentate ligands, where the transition metal is manganese, iron, cobalt, or nickel. Another object of the invention is to provide methods of making the transition metal-containing compounds of the present invention. A further object of the invention is to provide catalysts that comprise the transition metal-containing compounds of the invention, and methods of using these catalysts to catalyze the hydrogenation of olefins, including enantioselective hydrogenation of prochiral olefins.

These and other objects of the invention are, individually or combined accomplished with compounds represented by formula (I):

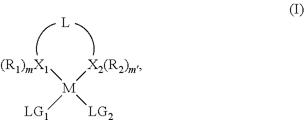

or salts thereof,

wherein

M represents a metal atom selected from the group consisting of manganese, iron, cobalt, and nickel;

L represents linking group selected from the group consisting of a substituted or unsubstituted, straight-chain or branched, saturated or unsaturated C₁₋₄₀ hydrocarbyl group; a substituted or unsubstituted, saturated or unsaturated C₃₋₄₀ (hetero)cyclohydrocarbyl group; a substituted or unsubstituted C₃₋₄₀ (hetero)aryl group; and a metallocene where each aromatic ring has at least one substituted or unsubstituted, straight-chain or branched, saturated or unsaturated C₁₋₄₀ hydrocarbyl group that connects to one of X₁ and X₂;

each of X₁ and X₂, individually, represents an atom selected from the group consisting of nitrogen, phosphorus, arsenic, oxygen, sulfur, and selenium, with the proviso that X₁ represents nitrogen, phosphorus, or arsenic when X₂ represents oxygen, sulfur, or selenium, or with the proviso that X₁ represents oxygen, sulfur, or selenium when X₂ represents nitrogen, phosphorus, or arsenic;

each of LG₁ and LG₂, individually, represents a leaving group;

each R₁ and R₂, individually, represents a hydrogen atom, a substituted or unsubstituted, straight-chain or branched, saturated or unsaturated C₁₋₄₀ hydrocarbyl group; a substituted or unsubstituted, saturated or unsaturated C₃₋₄₀ (hetero)cyclohydrocarbyl group; and a substituted or unsubstituted C₃₋₄₀ (hetero)aryl group; or a halogen atom, where at least one hydrogen atom from at least one carbon atom in the L group is optionally removed to form a (hetero)cycle with at least one R₁ group and at least one R₂ group; and

each of m and m′, individually, represents 0 or 1 when X₁ or X₂ represents an atom selected from the group consisting of oxygen, sulfur, and selenium, or represents 1 or 2 when X₁ or X₂ represents an atom selected from the group consisting of nitrogen, phosphorus, and arsenic.

### DETAILED DESCRIPTION OF THE INVENTION

Disclosed is a new composition of matter, specifically transition metal-containing dialkyl compounds with bidentate ligands of the form κ²-(X₁—X₂)M(LG₁)(LG₂)(X₁—X₂=bidentate ligand; M=Mn, Fe, Co, and/or Ni; each of LG₁ and LG₂=a leaving group). The bidentate ligand is also referred to as a bis-chelating ligand and is chiral or achiral. The methodology for making and using these transition metal-containing compounds disclosed herein is broad enough to include a number of bidentate chiral phosphines, including, but not limited to chiraphos, Duphos, and Duanphos. The disclosure also includes the application of these new transition metal-containing compounds in catalysts that are functional for the asymmetric hydrogenation of alkenes, an important synthetic route for preparing single enantiomer drugs, agrochemicals and fragrances. The asymmetric hydrogenation of unactivated olefins that lack directing groups is an unsolved problem. The disclosed transition metal-containing compounds are competent catalysts for such catalytic transformations. Other uses for these complexes that might be realized in the future are as transfer hydrogenation catalysts, hydroformylation catalysts, and olefin hydrosilylation catalysts.

The disclosed transition metal-containing compounds represent a novel route towards synthesizing many catalysts for these reactions. The method disclosed herein for producing the new transition metal-containing compounds requires few steps from commercially available precursors, involving reactions that are relatively atom economical and do not require halogenated or aromatic solvents. The disclosed compounds have been tested experimentally, and the present inventors have prepared and fully characterized transition metal-containing compounds and evaluated their catalytic performance. In general, the transition metal-containing compounds of the present invention exhibit high conversions and variable enantioselectivities in catalyzing the hydrogenation of olefins. The transition metal-containing compounds of the present invention are air and moisture sensitive, but can be handled under an inert (i.e., dinitrogen) atmosphere. In view of the foregoing discoveries by the present inventors, the present invention was made.

Methyl groups are shown in the representations of compounds included herein as “CH₃” or “Me”. Ethyl groups are shown in the representations of compounds included herein as “Et”. Iso-propyl groups and tert-butyl groups are shown in the representations of compounds included herein as “ⁱPr,” “iPr,” or “i-Pr” and “^{t}Bu,” “tBu,” or “t-Bu,” respectively. Phenyl groups are shown in the representations of compounds included herein as “Ph”. Last, “py” represents pyridine, and “Ns” represents neosilyl (neosilyl is represented by CH₂SiMe₃, so “Ns” also represents CH₂SiMe₃).

Unless stated otherwise, the compounds represented by formula (I) can be referred to as “a transition metal-containing compound represented by formula (I)” or “a compound represented by formula (I).” Further, the singular forms “a,” “an,” and “the” include plural referents unless the context clearly dictates otherwise. Thus, for example, reference to “a compound represented by formula (I)” could refer to at least two compounds, each represented by formula (I).

Unless otherwise stated, the terms “olefin” and “unsaturated substrate” used herein can be used interchangeably to refer one compound. For example, in the context of the present invention, ethylene can be described as an olefin or an unsaturated substrate.

The transition metal-containing compounds of the present invention are compounds represented by formula (I):

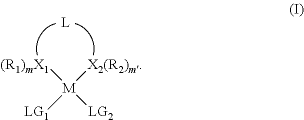

In this formula, M represents a metal atom selected from the group consisting of manganese, iron, cobalt, and nickel, where all valence states thereof are available. In preferred embodiments, M represents an iron, a cobalt, or a nickel atom. In particularly preferred embodiments, M represents a cobalt atom or a nickel atom. In the most preferred embodiments, M represents a cobalt atom.

The bidentate ligand that binds to the transition metal M is represented by (R₁)ₘX₁-L-X₂(R₂)ₘ, referred herein as the “X₁—X₂ ligand”. Each of X₁ and X₂ represents, individually, a Group 15 element selected from the group consisting of nitrogen, phosphorus, and arsenic or a Group 16 element selected from the group consisting of oxygen, sulfur, and selenium. When one of X₁ and X₂ represents a Group 16 element recited above, the other X group represents a Group 15 element recited above. In preferred embodiments, each of X₁ and X₂ represents a nitrogen atom, a phosphorus atom, or an arsenic atom. Further preferred embodiments include compounds where one of X₁ and X₂ represents an oxygen atom or a sulfur atom, and the other X atom represents a nitrogen atom or a phosphorus atom. In other preferred embodiments, each of X₁ and X₂ represents a nitrogen atom. In the most preferred embodiments, each of X₁ and X₂ represents a phosphorus atom.

L represents a group that links the X₁ and X₂ groups. Preferably, L represents linking group selected from the group consisting of a substituted or unsubstituted, straight-chain or branched, saturated or unsaturated C₁₋₄₀ (hetero)hydrocarbyl group; a substituted or unsubstituted, saturated or unsaturated C₃₋₄₀ (hetero)cyclohydrocarbyl group; a substituted or unsubstituted C₃₋₄₀ (hetero)aryl group; and a metallocene where each aromatic ring has at least one substituted or unsubstituted, straight-chain or branched, saturated or unsaturated C₁₋₄₀ (hetero)hydrocarbyl group that connects to one of X₁ and X₂. Each of these groups can have a heteroatom present within carbon-containing chains, rings, or groups thereof (the “(hetero)” modifier to these groups). Non-limiting examples for the heteroatom include nitrogen, phosphine, arsenic, oxygen, sulfur, selenium, silicon, and germanium. Further, each of these groups can be unsubstituted or substituted, where, in the substituted form, at least one hydrogen atom on the carbon chains or groups thereof is replaced with a substituting group. The substituting group is not particularly limited and can be (hetero)hydrocarbyl, (hetero)cyclohydrocarbyl, and (hetero)aryl groups defined above.

Non-limiting examples of the C₁₋₄₀ (hetero)hydrocarbyl group include alkylene groups of the formula —CₐH₂ₐ— where a is an integer (e.g. 1-100, all integers inclusive), with specific, non-limiting examples being methylene (X₁—CH₂—X₂), ethylene (X₁—CH₂—CH₂—X₂), n-propylene (X₁—CH₂—CH₂—CH₂—X₂), and n-butyl (X₁—CH₂—CH₂—CH₂—CH₂—X₂); alkenylene groups, which are acyclic carbon chains having at least one double carbon-carbon bond present in the chain, specific, non-limiting examples being ethenylene (X₁—C(H)═C(H)—X₂), 1-n-propenylene (X₁—CH₂═CH₂—CH₂—X₂), and 2-n-propenylene (X₁—CH₂—CH₂═CH₂—X₂). Terminal atoms of the substituted or unsubstituted, straight-chain or branched, saturated or unsaturated C₁₋₄₀ (hetero)hydrocarbyl group can bond to one or both of the X₁ and X₂ atoms through a multiple bond such as a double bond (e.g. X₁=C(H)—C(H)=X₂).

Non-limiting examples of the substituted or unsubstituted C₃₋₄₀ (hetero)cyclohydrocarbyl group include cyclobutyl, cyclopentyl, cyclohexyl, cycloheptyl, pyrrolidyl, piperidyl, phosphinanyl, tetrahydrofuryl, and tetrahydrothiophenyl. Non-limiting examples of the substituted or unsubstituted C₃₋₄₀ (hetero)aryl group in cyclopentadienyl, phenyl, naphthyl, anthracyl, phenanthrenyl, and pyridyl.

The C₃₋₄₀ (hetero)cyclohydrocarbyl and the C₃₋₄₀ (hetero)aryl group can be bound to the X₁ and X₂ atoms through substituted or unsubstituted, saturated or unsaturated, linear or branched C₁₋₂₀ (hetero)hydrocarbyl groups, which can also have heteroatoms defined herein in the main carbon chain of the group and can be substituted. Non-limiting examples of L groups having such a structure are as follows:

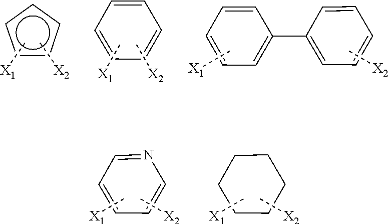

where the dashed lines represent the substituted or unsubstituted, saturated or unsaturated, linear or branched C₁₋₂₀ (hetero)hydrocarbyl groups defined above.

For the L groups that have an aromatic ring, complexes known as “sandwich complexes” or “metallocenes” can be formed. These complexes generally have a transition metal present between two effectively parallel aromatic rings. A common example of a sandwich complex, known to one of ordinary skill in the art, is ferrocene. In the context of the present invention, the transition metal of the sandwich complex, which can be iron, ruthenium, osmium, cobalt, rhodium, vanadium, chromium, manganese, cobalt, or nickel (all valences available), and the second aromatic ring that is present in the sandwich complex qualify as a substituting group for the substituted C₃₋₄₀ (hetero)aryl groups of the present invention. The preferred metal is iron.

Non-limiting examples of the aromatic rings for each ring of the sandwich complexes include aromatic rings such as cyclopentadienyl, phenyl, and naphthyl rings and heterocyclic aromatic rings such as pyridine, pyrrole, and oxepin. The aromatic rings of the sandwich moiety can be substituted or unsubstituted. The substituting groups are not particularly limited and can be any of the substituting groups disclosed in the entirety of this specification.

The aromatic rings of the sandwich complexes can be substituted with, e.g., a halogen atom, a (cyclo)(hetero)alkyl group, an amine group, a phosphine group, a (hetero)aryl group. The halogen atoms are those of Group 17 of the periodic table of elements, e.g., fluorine, chlorine, bromine, and iodine. Non-limiting examples of these sandwich complexes in the L group are shown below:

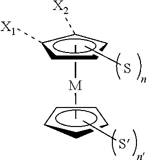

The dashed lines to X₁ and X₂ represent any of the linking groups defined herein. The X₁ and X₂ groups can be joined to any two of the carbon or heteroatoms of the aromatic ring, provided that each of X₁ and X₂ are not chemically bonded directly or through a hydrocarbyl group defined herein to the same atom of the aromatic ring.

Examples of the groups:

M: Fe, Co, Cr, Ni, and V;

S: CH₃, NH₂, PH₂, P(Ph)₂; SiMe₃;

S′: CH₃, NH₂, PH₂, P(Ph)₂; SiMe₃;

n: 0, 1, 2, or 3;

n′: 0, 1, 2, 3, 4, or 5.

L can also represent a metallocene where each aromatic ring has at least one substituted or unsubstituted, straight-chain or branched, saturated or unsaturated C₁₋₄₀ (hetero)hydrocarbyl group that connects to one of X₁ and X₂. The C₁₋₄₀ (hetero)hydrocarbyl group for this linking group is the same as above. The metals and the aromatic rings for the metallocene-linkers are preferably the same as those listed above for the sandwich complexes. Non-limiting examples of these metallocene linking groups are shown below:

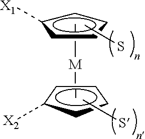

The dashed lines to X₁ and X₂ represent the C₁₋₄₀ (hetero)hyrdocarbyl group defined herein. The X₁ and X₂ groups can be joined to any of the carbon or heteroatoms of the aromatic rings, provided that each of X₁ and X₂ are not chemically bonded directly or through a hydrocarbyl group defined herein to the same atom of the aromatic ring.

Examples of the groups:

M: Fe, Co, Cr, Ni, and V;

S: CH₃, NH₂, PH₂, P(Ph)₂; SiMe₃;

S′: CH₃, NH₂, PH₂, P(Ph)₂; SiMe₃;

n: 0, 1, 2, 3, or 4;

n′: 0, 1, 2, 3, or 4.

Each of LG₁ and LG₂, individually, represents a leaving group. These group are not particularly limited so long as they are removed from the ligand sphere of the compound represented by formula (I) by, e.g., adding 0.5 to 5 molar equivalents, preferably 1 equivalent, of hydrogen (H₂) to the compounds. Without wishing to be bound to a particular theory, it is believed that the transition metal-containing compounds of the present invention release the leaving groups when the first equivalent of H₂ is introduced during the hydrogenation of olefins in the presence of the transition metal-containing compounds disclosed herein.

Each of LG₁ and LG₂ can, individually, represent pseudo-halides such as a triflate group, a tosylate group, and an acetate group. In preferred embodiments, at least one of LG₁ and LG₂ represents an acetate group. Each of LG₁ and LG₂ can, individually, also represent an activated halogen atom or halide. Activated halogen atoms/halides are those that have been activated by reaction with a reducing agent. Examples of halogen are identified above (e.g. fluorine, chlorine, bromine, and iodine), and the halide forms are those where one electron is added thereto (e.g. fluoride, chloride, bromide, and iodide). The halogen atoms can be reduced (activated) by reacting these atoms with a reducing agent. Non-limiting examples of the reducing agent include sodium triethylborohydride, metals such as zinc and magnesium, and alkyl lithium compounds such methyl lithium, ethyl lithium, n-butyl lithium, and (trimethylsilyl)methyllithium. Most preferably, however, each of LG₁ and LG₂ does not represent any of the halogen atoms, either in neutral or anionic (halide) form.

In preferred embodiments, each of LG₁ and LG₂ is a group represented by formula (II):

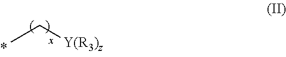

wherein Y represents an atom selected from the group consisting of carbon, nitrogen, oxygen, silicon, phosphorus, sulfur, germanium, arsenic and selenium; each R₃ group, individually, represents a hydrogen atom, a C₁₋₂₀ alkyl group optionally having at least one heteroatom, a C₃₋₂₀ (hetero)cyclohydrocarbyl group, a C₅₋₃₀(hetero)aryl group, or a halogen atom. When Y represents a carbon, silicon, or germanium atom, z represents an integer of 3. When Y represents a nitrogen, phosphorus, or arsenic atom, z represents an integer of 2. When Y represents an oxygen, sulfur or selenium atom, z represents an integer of 1. In some embodiments, each of LG and LG′ represents identical groups represented by formula (II). In other preferred embodiments, each of LG and LG′ represents different groups represented by formula (II). The symbol x represents the number of methylene groups that bind the atoms represented by Y metal M. The ranges for these numbers is from zero (0) to ten (10). The integers between these numbers are also included, which are one (1), two (2), three (3), four (4), five (5), six (6), seven (7), eight (8), nine (9), and ten (10). In particularly preferred embodiments, each of LG₁ and LG₂, individually, represents a neopentyl group or a neosilyl group, which are encompassed by formula (II). In the most preferred embodiments, each of LG₁ and LG₂ represents a neosilyl group.

Each of R₁ to R₃, individually, represents a hydrogen atom, a substituted or unsubstituted, straight-chain or branched, saturated or unsaturated C₁₋₄₀ (hetero)hydrocarbyl group; a substituted or unsubstituted, saturated or unsaturated C₃₋₄₀ (hetero)cyclohydrocarbyl group, and a substituted or unsubstituted C₃₋₄₀ (hetero)aryl group; or a halogen atom, where at least one hydrogen atom from at least one carbon atom in the L group is optionally removed to form a (hetero)cycle with at least one R₁ group and at least one R₂ group. Examples of the halogen atoms are the same as those described above. Specific, non-limiting examples of the C₁₋₄₀ (hetero)hydrocarbyl groups include methyl, ethyl, n-propyl, i-propyl, n-butyl, t-butyl, n-pentyl, neopentyl, n-hexyl, and cyclohexyl groups or additional groups recited above for L. Non-limiting examples for the substituted or unsubstituted, saturated or unsaturated C₃₋₄₀ (hetero)cyclohydrocarbyl group are the same as those defined above in L. Non-limiting examples of C₃₋₄₀(hetero)aryl group include, but are not limited to, tolyl, xylyl, phenyl, naphthalenyl, and additional examples listed above for L. The phenyl group is particularly preferred as the aryl group.

The C₃₋₄₀ (hetero)cyclohydrocarbyl group and the C₃₋₄₀(hetero)aryl group can be polycyclic (e.g. having one or more non-aromatic or aromatic rings), where the rings which may be fused, connected by single bonds or other groups.

For the heteroatoms that are optionally present in the LG₁ and/LG₂ groups, the heteroatoms are the same as those described above for linking group L.

In preferred embodiments, at least one of the R₁ to R₃ groups can be chemically bonded to an atom from the L group, such as a carbon atom, to form at least one ring structure. These rings can also be heterocycles, because the X₁ and/or X₂ atom can be included in the ring structure. These rings can be saturated or unsaturated (hetero)cyclic rings and/or can be fused with other rings such as aryl rings.

The symbols m and m′ represent the number of R₂ and R₃ groups bound to the element of X₁ and X₂, respectively. When X₁ and X₂ represent a Group 15 element, m and m′ are integers of 1 or 2, because double bonds can be formed between the X₁ and/or X₂ and the terminal atoms of the L group. When X₁ and X₂ represent a Group 16 element, each of m and m′ is 0 or 1. For those embodiments where one or both of m and m′ is 0, no R₁ and/or R₂ groups are bound to X₁ and X₂, respectively.

In one preferred embodiment, M represents a cobalt atom, each of X₁ and X₂ represents a phosphorus atom, L represents an ethylene group, each of m and m′ are equal to 2, each of LG₁ and LG₂ represents a group represented by formula (II) where each Y represents a silicon atom, each o represents an integer of 3, each R₃ represents a methyl group, and each n is equal to 1. Each of R₁ and R₂ represents a group defined above.

In another preferred embodiment, M represents a cobalt atom, each of X₁ and X₂ represents a nitrogen atom, L represents an ethylene group, each of m and m′ are equal to 2, each of LG₁ and LG₂ represents a group represented by formula (II) where each Y represents a silicon atom, each o represents an integer of 3, each R₃ represents a methyl group, and each n is equal to 1. Each of R₁ and R₂ represents a group defined above.

In another preferred embodiment, M represents a cobalt atom, each of X₁ and X₂ represents a nitrogen atom, L represents a group bound to X₁ and X₂ as follows: X₁═C(CH₃)—C(CH₃)═X₂ and therefore each of m and m′ represents an integer of 1, each of LG₁ and LG₂ represents a group represented by formula (II) where each Y represents a silicon atom, each o represents an integer of 3, each R₃ represents a methyl group, and each n is equal to 1. Each of R₁ and R₂ represents a group defined above.

In another preferred embodiment, M represents a cobalt atom, X₁ represents a nitrogen atom, X₂ represents a phosphorus atom, each of m and m′ represents an integer of 2, L represents an ethylene group, each of LG₁ and LG₂ represents a group represented by formula (II) where each Y represents a silicon atom, each o represents an integer of 3, each R₃ represents a methyl group, and each n is equal to 1. Each of R₁ and R₂ represents a group defined above.

In a further preferred embodiment, M represents a cobalt atom, X₁ represents a phosphorus atom, X₂ represents an oxygen atom, m represents an integer of 2, m′ represents an integer of 1, L represents an ethylene group, each of LG₁ and LG₂ represents a group represented by formula (II) where each Y represents a silicon atom, each o represents an integer of 3, each R₃ represents a methyl group, and each n is equal to 1. Each of R₁ and R₂ represents a group defined above.

In a further preferred embodiment, M represents a cobalt atom, X₁ represents an oxygen atom, X₂ represents a phosphorus atom, m represents an integer of 1, m′represents an integer of 2, L represents an ethylene group, each of LG₁ and LG₂ represents a group represented by formula (II) where each Y represents a silicon atom, each o represents an integer of 3, each R₃ represents a methyl group, and each n is equal to 1. Each of R₁ and R₂ represents a group defined above.

The particularly preferred X₁-X₂ ligands of the present invention include, but are not limited to:
1,2-bis(diphenylphosphino)ethane (“dppe”);
1,2-bis(diethylphosphino)ethane (“depe”);
(2S,3S)-(−)-bis(diphenylphosphino)butane (“chiraphos”);
(−)-1,2-Bis([(2R,5R)-2,5-dialkylphospholano]benzene (“duphos”) where each alkyl group is a methyl group, an ethyl group, or an isopropyl group;
(1R,1′R,2S,2′S)-2,2′-di-tert-butyl-2,3,2′,3′-tetrahydro-1H,1′H-(1,1′)biisophosphindolyl(“duanphos”);
(N,N′E,N,N′E)-N,N′-(butane-2,3-diylidene)bis(2,6-diisopropylaniline) (“iPrDI”);
(1R,1′R,2R,2′R)-(−)-2,2′diphenylphosphino-1,1′-bicyclopentyl (“(R,R)-BICP”);
(S)-(6,6′-dimethoxybiphenyl-2,2′-diyl)bis[bis(3,5-di-tert-butyl-4-methoxyphenyl)phosphine] (“SL-A109-2”);
(R)-(+5,5′-bis[di(3,5-di-tert-butyl-4-methoxyphenyl)phosphine]-4,4′-bi-1,3-benzodioxole (“(R)-DTBM-SegPhos”);
(R)-(+)-5,5′-Bis[di(3,5-xylyl)phosphino]-4,4′-bi-1,3-benzodioxole (“(R)-DM-SegPhos”);
(S)-2,2′-bis[di-3,5-xylyl)phosphino]-6,6′-dimethoxy-1,1′-biphenyl (“SL-A120-2” or “(S)-DM-MeOBIPHEP”);
(R)-2,2′-bis(di-p-tolylphosphino)-6,6′-dimethoxy-1,1′-biphenyl (“SL-A102-1”);
(S)-4-tert-butyl-2-[(S)-2-(bis(1-phenyl)-phosphino)ferrocen-1-yl]oxazoline (“SL-N004-2”);
(R)-1-[(S)-2-(di-1-naphtylphosphino)ferrocenyl]-ethyl-di-3,5-xylylphosphine (“SL-J404-1”);
(R)-1-[(Sp)-2-(diphenylphosphino)ferrocenyl]ethyldicyclohexylphosphine (“SL-J001-1”);
(2R)-1-[(1R)-1-[bis(cyclohexyl)phosphino]ethyl]-2-(diphenylphosphino)-1′-[(1R)-1-[bis(cyclohexyl)phosphino]ethyl]-2′-(diphenylphosphino)ferrocene (“SL-J851-2”);
(3S,3′S,4S,4′S,11bS,11′bS)-(+)-4,4′-di-tert-butyl-4,4′,5,5′-tetrahydro-3,3′-bi-3H-dinaphtho[2,1-c:1′,2′-e]phosphepin (“(S)-Binapine”);
(2S,5S)-1-(2-(bis(3,5-di-tert-buty-4-methoxyphenyl)phosphino)phenyl)-2,5-dimethylphospholane (“(S,S)-Me-UCAP-DTBM”);
(R)-1-[(S_{P})-2-(di-tert-butylphosphino)ferrocenyl]ethylbis(2-methylphenyl)phosphine (“JosiPhos” or “SL-J505-1”);
(R)-1-[(S)-2-di-(4-methoxy-3,5-dimethylphenyl-phosphino)ferrocenyl]-ethyl-di-3,5-xylylphosphine (“SL-J418-1”);
(1R)-1-(diphenylphosphino)-2-[(1R)-1-[(diphenylphosphino)methylamino]ethyl]-1′-(diphenylphosphino)-2′-[(1′R)-1′-[(diphenylphosphino)methylamino]ethyl]-ferrocene (“SL-F011-2”);
(R)-1-{(S_{P})-2-[bis[4-(trifluoromethyl)phenyl]phosphino]ferrocenyl}ethyldi-tert-butylphosphine (“SL-J011-1”);
(2R)-1-[(1R)-1-[bis(3,5-dimethylphenyl)phosphino]ethyl]-2-(diphenylphosphino)ferrocene (“SL-J005-1”); and
(4S,4′S)-4,4′-dimethyl-4,4′,5,5′-tetrahydro-2,2′-bioxazole (“pybox”).

Ligand SL-A109-2 is the most preferred ligand. These ligands and other preferred ligands are represented in Scheme 1:

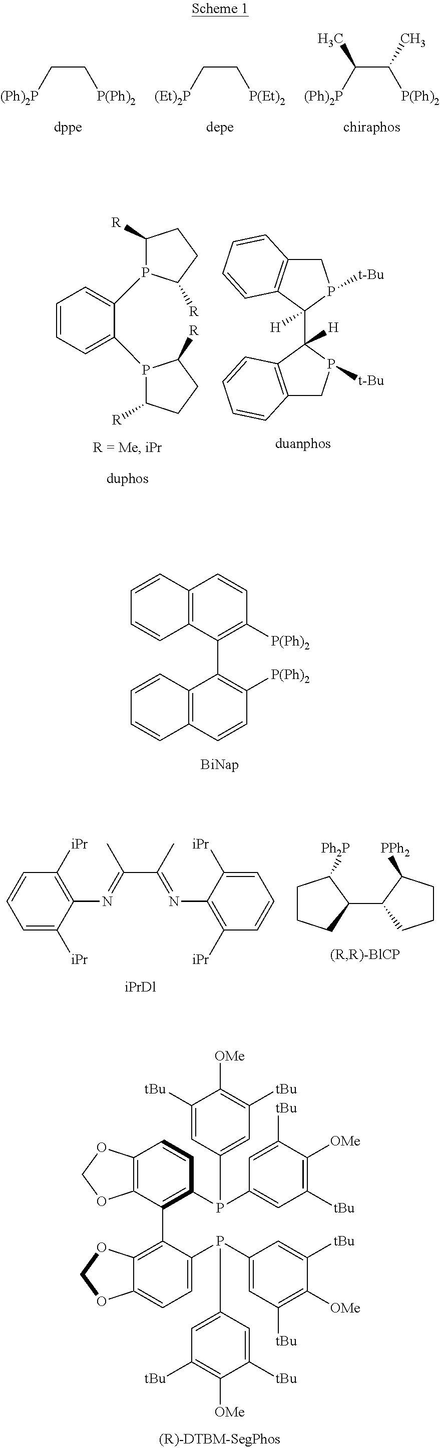

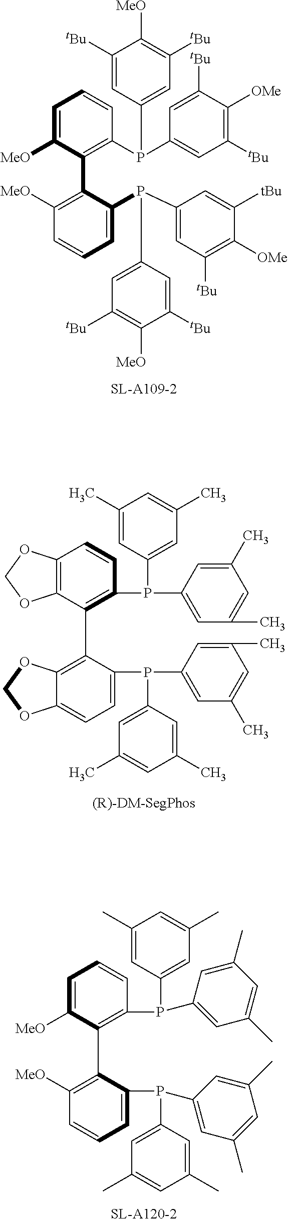

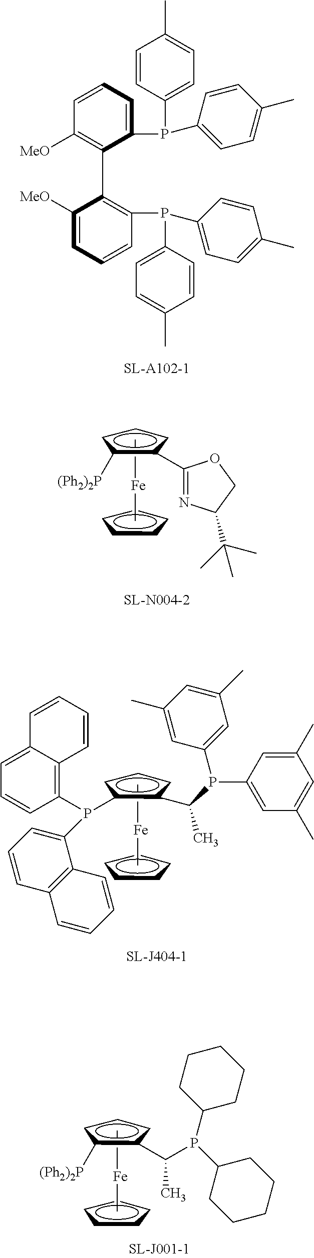

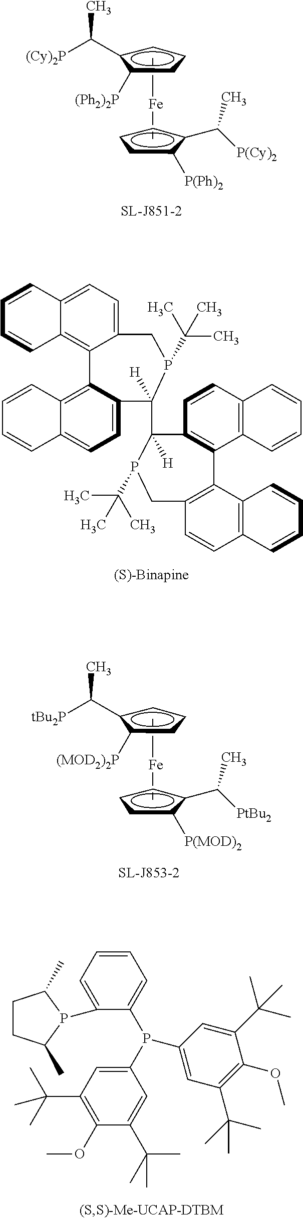

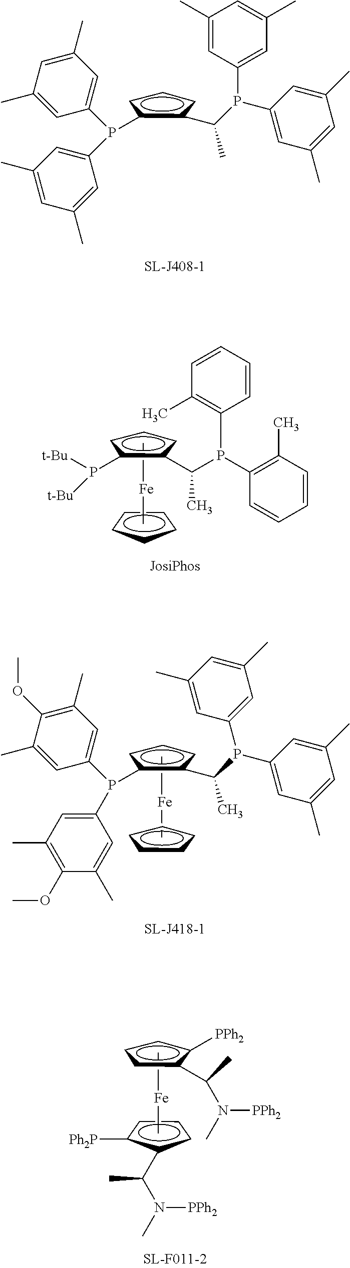

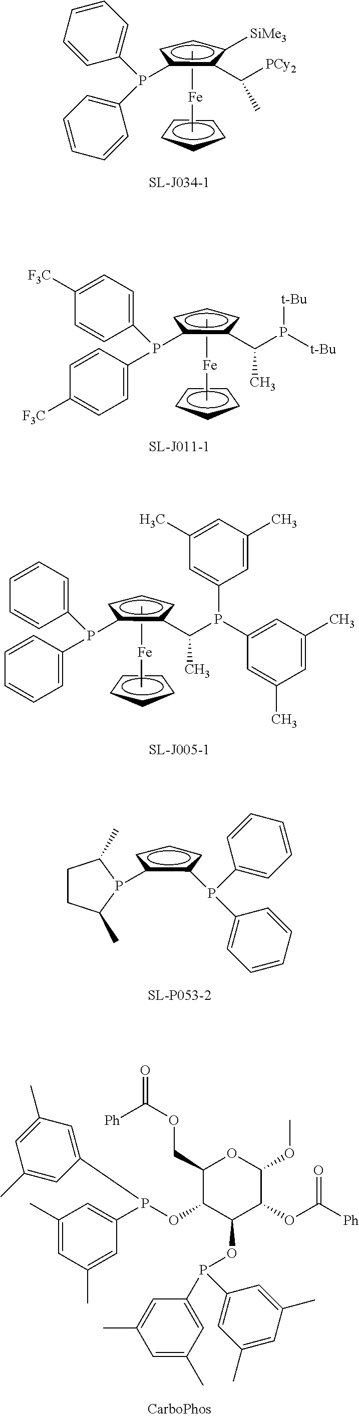

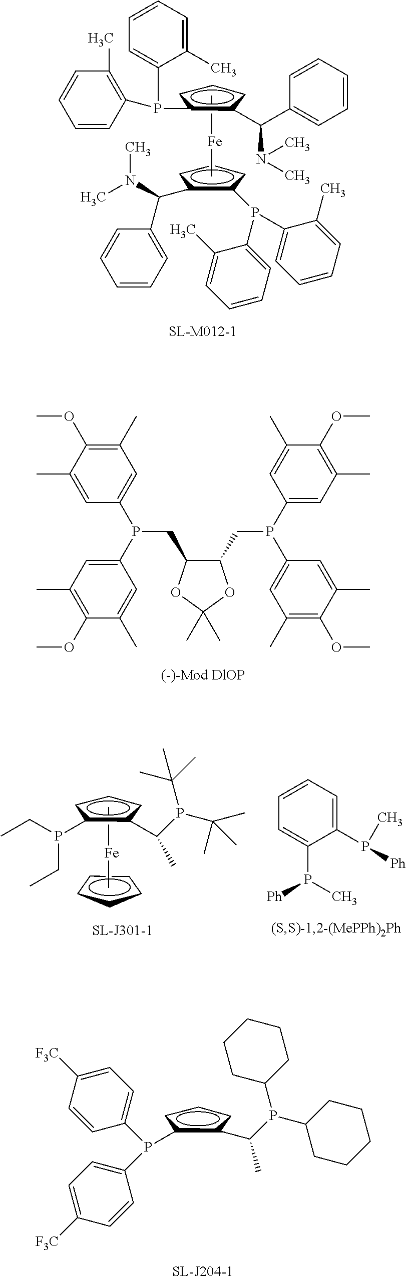

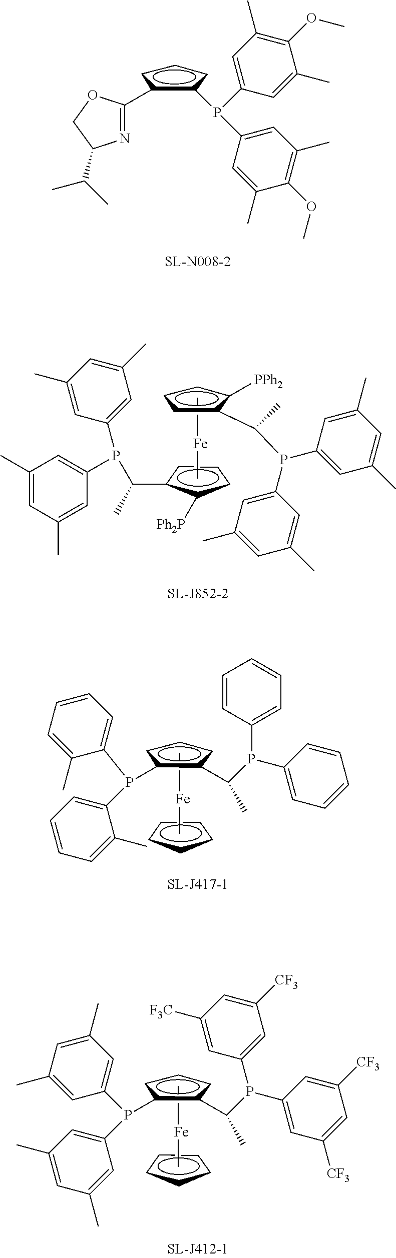

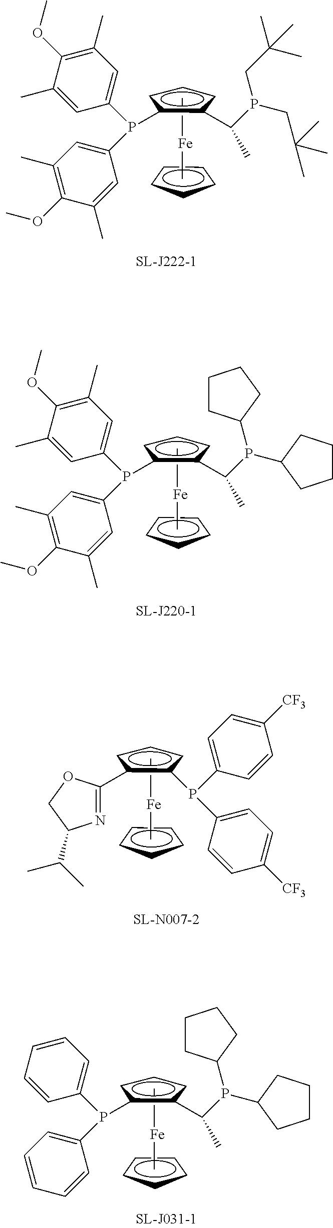

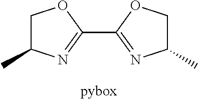

Each “MOD” in SL-J853-2 represents a 3,5-dimethyl-4-methoxyphenyl group.

Without wishing to be bound to a particular theory, it is believed that the bidentate ligands are likely bound to the metal atom (e.g. manganese, iron, cobalt, or nickel) through dative bonds. Lone pairs of electrons from, e.g., the phosphorus atom of the bidentate ligand, fill an unoccupied atomic orbital of the transition metal. This bonding scheme can also be described as σ-donation. Each of the Group 15 element and Group 16 element that is present in the bidentate ligand likely binds to the transition metal through dative bonds, thereby chelating to the transition metal. In regard to the bonding between the transition metal and the carbon or silicon atoms of the remaining ligands, it is believed that these bonds are more covalent in nature.

Preferred, non-limiting compounds of the invention are shown in Scheme 2.

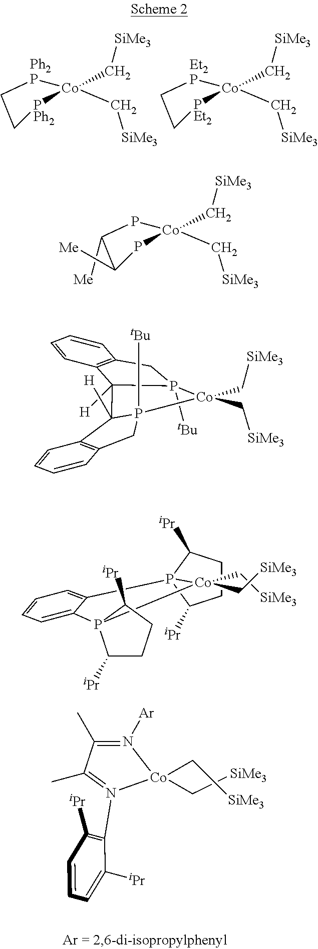

The present invention also includes methods of making the compounds of formula (I). One method comprises reacting the X₁-X₂ ligand with a metal M source such as [(py)₂M(LG₁)(LG₂)], M is a metal as defined above, each of LG₁ and LG₂ are as defined above and “py” represents pyridine. An example of the metal M source is [(py)₂Co(CH₂SiMe₃)₂] (see Zhu, Janssen, and Budzelaar, above). In this method, the metal M source is deposited in a reaction vessel such as a glass round-bottom flask in a solvent capable of dissolving the metal M source, and the depositing is carried out at a reduced temperature such as −60° C. The solvent is not particularly limited so long as it solvates the reactants, and is, e.g., benzene or diethyl ether. After this step, the X₁−X₂ ligand is added to the reaction vessel in an amount that is at least half the molar amount of the metal M source (e.g. molar ratio [M]/[(X₁−X₂ ligand)]≧0.5 where M is the metal from the metal source, [M] is the molar amount of the metal, and [(X₁−X₂ ligand)] is the molar amount of the X₁−X₂ ligand). A preferred range for the [M]/[(X₁−X₂ ligand)] ratio is from 0.5 to 2. More preferably, this range is from 0.5 to 1. In the most preferred embodiments, the metal M source and the X₁−X₂ ligand are present in equimolar amounts (e.g. molar ratio [M]/[(X₁−X₂ ligand)] equals 1). The temperature of the reaction between a X₁−X₂ ligand and the metal M source is not particularly limited so long as the reaction proceeds, but the temperature of the reaction should not exceed 40° C. Preferably, the temperature ranges from −60° C. to 40° C., even more preferably from −40° C. to room temperature, and all intervening integers are included. This reaction can also be carried out at a temperature gradient where the temperature is changed, preferably within these ranges, while the reaction is carried out. The reaction product can be isolated by known methods.

The present invention also relates to catalysts that comprise the transition metal-containing compounds of the invention. The catalyst of the present invention comprises at least one of the transition metal-containing compounds represented by formula (I). In some embodiments of the present invention, the catalysts comprise additional components, such as solvents and supports, so long as the transition metal-containing compounds are present in the catalyst in an amount effective for catalyzing the hydrogenation of olefins. Preferably, the total amount of the transition metal-containing compounds represented by formula (I) present in the catalyst is from 0.5 to 10 mole %, relative to the total moles of the olefin.

In another embodiment of the present invention, the catalyst can be made in situ. Here, the metal M source, a X₁−X₂ ligand, an unsaturated substrate, hydrogen, and a solvent are placed in a reaction vessel simultaneously or in any order, and a compound represented by formula (I) is formed in the presence of the olefin and hydrogen, thereby generating the catalyst in situ. The hydrogenation of the unsaturated substrate can proceed once the compound represented by formula (I) has been made in situ. Preferably the metal M source, X₁−X₂ ligand, and the unsaturated substrate are added to a reaction vessel at a temperature of −60° C. to 40° C. The hydrogen is then introduced and proceeds according to the hydrogenation conditions discussed below.

In another embodiment of the present invention, the catalyst can be made in situ through a different method. Here, the metal M source such as [M(LG₁)(LG₂)] is used, where each of LG₁ and LG₂, individually, is a halide or a pseudo-halide. An example of the metal M source is CoCl₂. Here, the metal M source is first reacted with a X₁−X₂ ligand for a reaction time of at least ten minutes at a reaction temperature of at least room temperature, and the complex product is then added to the reaction vessel with addition of the reducing agent, preferably (trimethylsilyl)methyllithium, an unsaturated substrate defined herein, hydrogen, and a solvent simultaneously or in any order, and a compound represented by formula (I) is formed in the presence the unsaturated substrate (e.g. an olefin disclosed herein) and hydrogen. The catalyst is thus generated in situ. The hydrogenation of the unsaturated substrate can proceed once a compound represented by formula (I) has been made in situ. Preferably, the metal M source, the X₁−X₂ ligand, and the unsaturated substrate are added to a reaction vessel at a temperature of −60° C. to 40° C. The hydrogen is then introduced and proceeds according to the hydrogenation conditions discussed below.

In other embodiments of the invention, the present catalysts consist essentially of at least one at least one of the transition metal-containing compounds disclosed herein and components that do not materially affect the basic and novel characteristics of the catalysts disclosed herein, such as inert impurities that inevitably form during synthesis of these transition metal-containing compounds. In other embodiments of the present invention, the catalysts consist of the transition metal-containing compounds disclosed herein.

The solvent of the catalyst is not particularly limited, as long as the solvent is capable of dissolving the olefin and the transition metal-containing compounds represented by formula (I). An example of the solvent is benzene. The support is also not particularly limited, so long as these compounds are supported thereby. It can be, for example, silica or resin beads.

The transition metal-containing compounds represented by formula (I) are efficient in hydrogenating olefins, particularly prochiral olefins, which, once hydrogenated, have at least one chiral carbon atom in the molecule. The hydrogenation reactions can be carried out by charging a thick walled glass vessel with an olefin and a transition metal-containing compound represented by formula (I). The amount of olefin (also referred herein as the substrate unless otherwise noted) is not particularly limited so long as the reaction proceeds. In preferred embodiments, the olefin is present in the glass vessel in an amount of 0.04 M to 1.0 M, more preferably from 0.09 to 1.0 M. In preferred embodiments, the olefin is present in an amount of about 0.8 M. In other preferred embodiments, the olefin is present in an amount of 0.04 M. A solvent is added to the glass vessel, and the atmosphere in the glass vessel is evacuated and replaced with hydrogen (H₂) at low temperatures, e.g. 80 K. The pressure of H₂ in the glass vessel is from sub-atmospheric pressure to 100 psi, preferably from atmospheric pressure to 100 psi. For higher pressures, a metal high-pressure reactor can be used. The pressure of hydrogen in the metal high-pressure reactor is from 100 psi to 1,000 psi, preferably from 100 psi to 500 psi. The temperature of this reaction is not particularly limited, so long as it is sufficient to carry out the hydrogenation of the olefins. A preferred range for the reaction temperature is −20° C. to 50° C. All intervening integers are included. Most preferably, the hydrogenation reactions are carried out at about room temperature.

In preferred embodiments, the catalyst further comprises an additive that increases the conversion of the olefins in the hydrogenation reactions. The additive preferably comprises a nitrogen-containing heterocycle, such as pyridine, 2-methylpyridine, 4-dimethylaminopyridine (DMAP), 4-methoxypyridine, and 4-tert-butylpyridine. The additive comprises one or more of these nitrogen-containing heterocycles. In other preferred embodiments, the additive comprises at least one imine that has a chiral moiety. In the most preferred embodiments, the additive comprises pyridine in an amount sufficient to accelerate the hydrogenation of the olefins.

In other preferred embodiments, free phosphines (e.g. PMe₃) are not present in the catalyst. Without wishing to be bound to a particularly theory, it is believed that free phosphines inhibit the catalytic ability of the compounds of the present invention in catalyzing the hydrogenation of prochiral olefins. Evidence for this effect has been reported in each of Hendrikse and Coenen and Hendrikse et al. In particularly preferred embodiments, the catalysts of the present invention comprise at least one compound represented by formula (I) and the additive, where no additional phosphine is present in the catalyst.

The amount of the additive present in the catalyst is not particularly limited so long as it is present in an amount effective for increasing the conversion of the olefin. Preferably, the additive is present in the catalysts in an amount of 0.1 to 5 molar equivalents, more preferably 0.5 to 2 molar equivalents, most preferably about an equimolar equivalent, relative to the molar amount of the compounds represented by formula (I).

The catalysts of the invention are efficient in hydrogenating olefins, preferably prochiral olefins which, once hydrogenated, have at least one chiral carbon atom in the molecule. The olefins of the catalyzed hydrogenation reactions are not particularly limited. By way of example, (R)-propane-1,2-diyldibenzene and (S)-propane-1,2-diyldibenzene result from the hydrogenation of E-α-methylstilbene ((E)-prop-1-ene-1,2-diyldibenze), shown below, where the star indicates the chiral carbon atom:

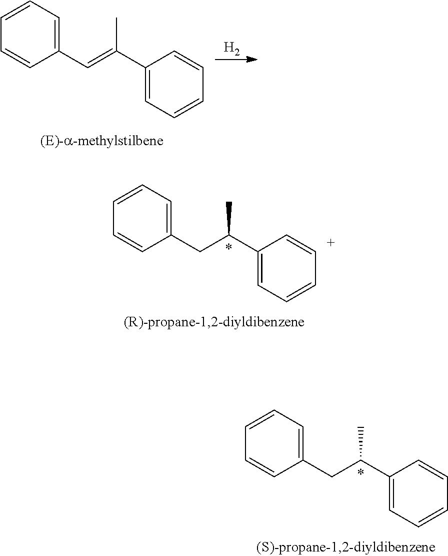

The following non-limiting examples are intended to illustrate the present invention. Unless otherwise stated, the temperatures for the reactions are reported in degrees centigrade and all pressures are reported in atmospheres.

### EXAMPLES

### Synthesis Example 1

### Synthesis of Compound A (dppeCoNs₂)

A 100 mL round bottom flask was charged with 40 mL diethyl ether, a stir bar, and (py)₂CoNs₂ (161 mg, 0.41 mmol), and cooled to about −60° C. A scintillation vial was charged with 10 mL dppe (164 mg, 0.41 mmol) and cooled to −35° C. The solution of dppe was added to the round bottom flask, which was then warmed to room temperature. The reaction solution in the round bottom flask turned from dark green to orange-red. After stirring at room temperature for 2 hours, the reaction solution was evaporated to dryness, reconstituted with 15 mL of diethyl ether and filtered through celite. The filtrate was further concentrated to about 3 mL, diluted with 5 mL pentane and stored overnight at −35° C. to afford orange crystals of dppeCoNs₂ in two crops (226 mg, 87% overall yield). Magnetic susceptibility (C₆D₆, 293 K, Evans): μ_{eff}=1.9μ_{B}. ¹H NMR (C₆D₆, 400 MHz): 30.3 (2100 Hz), 5.64 (56 Hz), 3.38 (52 Hz), 0.0 (76 Hz), −0.71 (223 Hz), −14.7 (1800 Hz).

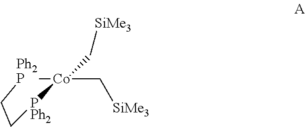

### Synthesis Example 2

### Synthesis of Compound B (depeCoNs₂)

A 100 mL round bottom flask was charged with 40 mL diethyl ether, a stir bar, and (py)₂CoNs₂ (206 mg, 0.53 mmol), and cooled to about −60° C. A scintillation vial was charged with 10 mL depe (109 mg, 0.53 mmol) and cooled to −35° C. The solution of depe was added to the round bottom flask, which was then warmed to room temperature. The reaction solution in the round bottom flask turned from dark green to orange. After stirring at room temperature for 2 hours, the reaction solution was evaporated to dryness, reconstituted with 15 mL of diethyl ether and filtered through celite. The filtrate was evaporated and washed with cold pentane and further dried to afford an orange solid (159 mg, 69%) of depeCoNs₂. ¹H NMR (C₆D₆, 400 MHz): 28.43 (393 Hz), 8.53 (16 Hz), 7.03 (20 Hz), 6.67 (16 Hz), 1.69 (161 Hz), −9.26 (165 Hz), −10.33 (br s, coincidental overlap), −12.70 (440 Hz).

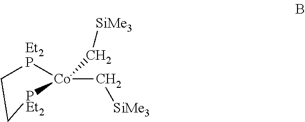

### Synthesis Example 3

### Synthesis of compound C ((2S,3S)-chiraphosCoNs₂)

A 100 mL round bottom flask was charged with 40 mL diethyl ether, a stir bar, and (py)₂CoNs₂ (70 mg, 0.18 mmol), and cooled to about −60° C. A scintillation vial was charged with 10 mL (2S,3S)-chiraphos (76 mg, 0.18 mmol) and cooled to −35° C. prior to addition. The (2S,3S)-chiraphos suspension was added to the round bottom flask, which was then warmed to room temperature. The reaction solution in the round bottom flask turned from dark green to orange-red. After stirring at room temperature for 2 hours, the reaction solution was evaporated to dryness, reconstituted with 15 mL of diethyl ether and filtered through celite. The filtrate was evaporated and washed with cold pentane and further dried to afford an orange solid (92 mg, 78%) of (2S,3S)-chiraphosCoNs₂. Magnetic susceptibility (C₆D₆, 293 K, Evans): μ_{eff}=1.6μ_{B}. ¹H NMR (C₆D₆, 300 MHz): 16.8 (142 Hz), 6.91 (43 Hz), 4.99 (28 Hz), 0.68 (46 Hz), −0.27 (209 Hz), −3.0 (71 Hz). Anal. Calcd. (C₃₆H₅₀CoP₂Si₂): C, 65.53; H, 7.64%. Found: C, 65.70; H, 7.62%.

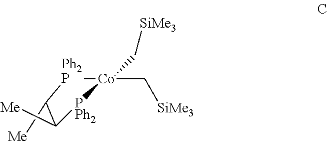

### Synthesis Example 4

### Synthesis of Compound D ((2R,5R)-ⁱPr-duphosCoNs₂)

A 100 mL round bottom flask was charged with 40 mL diethyl ether, a stir bar, and (py)₂CoNs₂ (314 mg, 0.80 mmol), and cooled to about −60° C. A scintillation vial was charged with 10 mL (2R,5R)-ⁱPr-duphos (336 mg, 0.80 mmol) and cooled to −35° C. The (2R,5R)-ⁱPr-duphos suspension was to the round bottom flask, which was then warmed to room temperature. The reaction solution in the round bottom flask turned from dark green to orange-red. After stirring at room temperature for 2 hours, the reaction solution was evaporated to dryness, reconstituted with 15 mL of diethyl ether and filtered through celite. The filtrate was evaporated and washed with cold pentane and further dried to afford an orange solid (490 mg, 94%) of (2R,5R)-ⁱPr-duphosCoNs₂. Magnetic susceptibility (C₆D₆, 293 K, Evans): μ_{eff}=2.0μ_{B}. ¹H NMR (C₆D₆, 400 MHz): 25.80 (114 Hz), 16.08 (30 Hz), 7.50 (12 Hz), 6.32 (68 Hz), 2.22 (173 Hz), 1.27 (54 Hz), 1.21 (d), 1.11 (d), 0.72 (d), −6.68 (214 Hz), −13.84 (104 Hz), −14.80 (166 Hz), −17.12 (85 Hz), −29.85 (627 Hz), −34.19 (689 Hz), −44.90 (614 Hz).

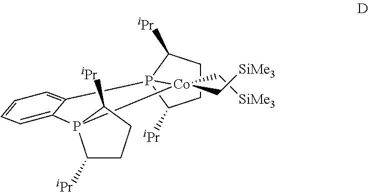

### Synthesis Example 5

### Synthesis of compound E ((1R,1′R,2S,2′S)-duanphosCoNs₂)

A 100 mL round bottom flask was charged with 40 mL diethyl ether, a stir bar, and (py)₂CoNs₂ (208 mg, 0.53 mmol), and cooled to about −60° C. A scintillation vial was charged with 10 mL (1R,1′R,2S,2′S)-duanphos (203 mg, 0.53 mmol) and cooled to −35° C. The (1R,1′R,2S,2′S)-duanphos suspension was added to the round bottom flask, which was then warmed to room temperature. The reaction solution in the round bottom flask turned from dark green to orange-red. After stirring at room temperature for 2 hours, the reaction solution was evaporated to dryness, reconstituted with 15 mL of diethyl ether and filtered through celite. The filtrate was evaporated and washed with cold pentane and further dried to afford an orange solid (304 mg, 93%) of (1R,1′R,2S,2′S)-duanphosCoNs₂. Magnetic susceptibility (C₆D₆, 293 K, Evans): μ_{eff}=1.7μ_{B}. ¹H NMR (C₆D₆, 400 MHz): 29.4 (290 Hz), 21.4 (74 Hz), 10.50 (21 Hz), 6.77 (19 Hz), 6.33 (151 Hz), 2.67 (42 Hz), −5.28 (137 Hz), −11.78 (321 Hz). Anal. Calcd. (C₃₂H₅₄CoP₂Si₂): C, 62.41; H, 8.84%. Found: C, 62.34; H, 8.45%.

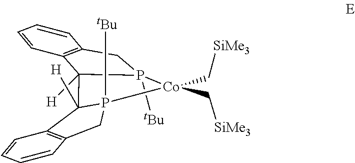

### Synthesis Example 6

### Synthesis of Compound F (iPrDICoNs₂)

A 100 mL round bottom flask was charged with 40 mL diethyl ether, a stir bar, and (py)₂CoNs₂ (61 mg, 0.16 mmol), and cooled to about −60° C. A scintillation vial was charged with 10 mL 1,2-dimethyl-1,2-di(arylimine) (aryl=2,6-diisopropylphenyl) (iPrDI, 63 mg, 0.16 mmol) and cooled to −35° C. The solution of iPrDI was added to the round bottom flask, which was then warmed to room temperature. The reaction solution in the round bottom flask turned from dark green to purple. After stirring at room temperature for 2 hours, the reaction solution was evaporated to dryness, reconstituted with 5 mL of pentane and filtered through celite. The filtrate was dried to afford a purple solid (87 mg, 88%) of iPrDiCoNs₂. Magnetic susceptibility (C₆D₆, 293 K, Evans): μ_{eff}=1.9μ_{B}. ¹H NMR (C₆D₆, 400 MHz): 4.09 (125 Hz), 2.88 (40 Hz), 2.15 (31 Hz), 1.18 (40 Hz), 0.61 (37 Hz), −0.87 (191 Hz), −9.69 (81 Hz), −21.89 (148 Hz).

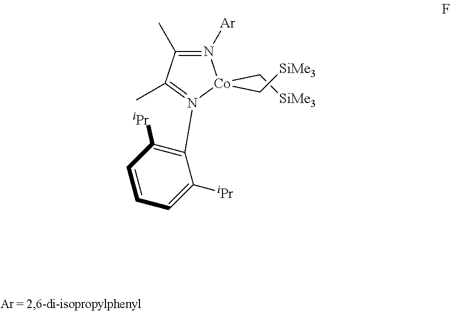

### Example 1

### Hydrogenation of (R)-1-methyl-4-(prop-1-en-2-yl)cyclohex-1-ene (α-Limonene) in the Presence of Compound A

α-Limonene and κ²-dppe(Co)(CH₂SiMe₃)₂, olefin I and compound A, respectively as shown below in Scheme 3, were placed in a reaction vessel and dissolved in benzene at 25° C. Hydrogen (H₂) was introduced into the reaction vessel at a pressure of four atmospheres, and hydrogenation of this olefin was carried out at 25° C. Compound A is the catalyst for this hydrogenation. The products obtained from this hydrogenation were (R)-4-isopropyl-1-methylcyclohex-1-ene (II) and 1-isopropyl-4-methylcyclohexane (III), where hydrogenation of the two carbon-carbon double bonds in the olefin was monitored for each carbon-carbon double bond and measured at intervals of 3 hours, 6 hours, and 12 hours.

The conversion percentages are included in the Scheme 3, shown below:

Scheme 3

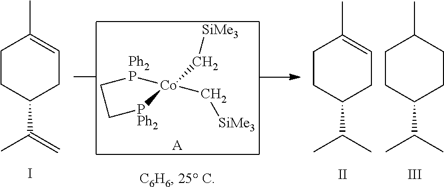

| | Product Distribution | |
|---|---|---|
| Conversion | (partial/full) | |
| 3 hr: 92% conversion | 45% | 47% |
| 6 hr: 97% conversion | 42% | 55% |
| 12 hr: 99% conversion | 48% | 51% |

As shown in Scheme 3, compound A successfully converted (R)-1-methyl-4-(prop-1-en-2-yl)cyclohex-1-ene to (R)-4-isopropyl-1-methylcyclohex-1-ene and 1-isopropyl-4-methylcyclohexane. The product distribution favored 1-isopropyl-4-methylcyclohexane at each time interval. However, the product distribution varied over time and was therefore inconsistent, unlike the overall conversion rate.

### Example 2

### Hydrogenation of E-α-methylstilbene in the Presence of Compound A

E-α-methylstilbene and compound A were placed in a reaction vessel and dissolved in toluene at 25° C. Hydrogen (H₂) was introduced into the reaction vessel at a pressure of four atmospheres, and hydrogenation of this olefin was carried out at 25° C. Compound A was the catalyst for this hydrogenation. The product obtained from this hydrogenation was propane-1,2-diyldibenzene, where conversion of this olefin to the product was measured at intervals of 1 hour, 3 hours, 6 hours, 12 hours, and 48 hours. The conversion data is shown below in Table I:

TABLE I

| Time (hr) | Conversion percentage (%) |
|---|---|
| 1 | 61 |
| 3 | 73 |
| 6 | 76 |
| 12 | 89 |
| 48 | 99 |

### Example 3

### Hydrogenation of E-α-methylstilbene in the Presence of Compound A and an Additive

The same experiment of Example 2 was carried here except that 5 mol % of the additive pyridine was added to the reaction vessel. After 1 hour and 3 hours of reaction time, the conversion percentages were 99% and 100%, respectively.

### Example 4

### Hydrogenation of E-α-methylstilbene in the Presence of Compound A and an Additive in Different Amounts

The same experiment of Example 2 was carried three additional times except that the additive pyridine was added to the reaction vessel in amounts of 2.5 mol %, 5 mol % and 25 mol %, respectively. The conversion percentages were measured for each of these three additional hydrogenation reactions 1 hour into the reactions and were found to be 65%, 99% and 99%, respectively.

### Example 5

### Hydrogenation of α-Limonene in the Presence of Compound C

The same reaction of Example 1 was carried out, except that compound C was used as the catalyst. After 12 hours, the conversion percentage of 99% was achieved. The product distribution favored (R)-4-isopropyl-1-methylcyclohex-1-ene in an amount of 73%.

### Example 6

### Hydrogenation of E-α-methylstilbene in the Presence of Compound C, Conversion Rates Measured at Different Times

E-α-methylstilbene and compound C were placed in a reaction vessel and dissolved in benzene at 25° C. Compound C was the catalyst for this hydrogenation. Hydrogen (H₂) was introduced into the reaction vessel at a pressure of four atmospheres, and hydrogenation of this olefin was carried out at 25° C. After twelve hours the conversion percentage was 97% with an enantiomeric excess (% ee) of 34%.

### Example 7

### Hydrogenation of Four Ethylenically Unsaturated Compounds in the Presence of Compound C

Four ethylenically unsaturated compounds (“substrate” in the following Table II) were hydrogenated with hydrogen in the presence of compound C, and the results are shown below in Table II. In reaction vessels, 0.84M of the ethylenically unsaturated compound to be hydrogenated and 5 mol % of compound C were dissolved in 0.74 mL of benzene. Hydrogen (H₂) was introduced into the reaction vessels at a pressure of four atmospheres, and hydrogenation of this olefin was carried out at 25° C.

TABLE II

| Substrate | Product | Conversion^{a} | % ee | time |
|---|---|---|---|---|
| 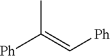 | 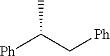 | 91% | 34%^{b} | 12 hr |
| 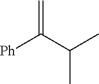 | 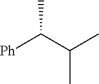 | 30% | 9%^{c,d} | 12 hr |
| 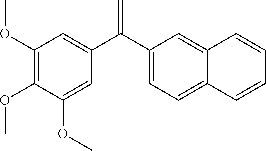 [SMILES: C=C(c1cc(OC)c(OC)c(OC)c1)c1ccc2ccccc2c1] | 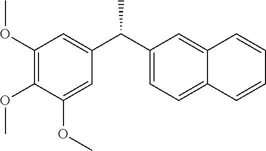 [SMILES: COc1cc([C@H](C)c2ccc3ccccc3c2)cc(OC)c1OC] | >98% | 20%^{b} | 12 hr |
| 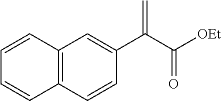 [SMILES: C=C(C(=O)OCC)c1ccc2ccccc2c1] | 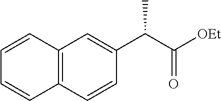 [SMILES: CCOC(=O)[C@@H](C)c1ccc2ccccc2c1] | 98%^{e} | 28%^{b} | 24 hr |

^{a}Conversions determined by GC-FID
^{b}% ee determined by chiral SFC-HPLC
^{c}% ee determined by chiral GC-FID
^{d}(R enantiomer)
^{e}Catalysis run at 0.1M [substrate]

### Example 8

### Hydrogenation of Five Ethylenically Unsaturated Compounds in the Presence of Compound D

Five ethylenically unsaturated compounds (“substrate” in the following Table III) were hydrogenated with hydrogen in the presence of compound D, and the results are shown below in Table III. In reaction vessels, 0.84M of the ethylenically unsaturated compound to be hydrogenated and 5 mol % of compound D were dissolved in 0.74 mL of benzene. Hydrogen (H₂) was introduced into the reaction vessels at a pressure of four atmospheres, and hydrogenation of this olefin was carried out at 25° C.

TABLE III

| Substrate | Product | Conversion^{a} | % ee | time |
|---|---|---|---|---|
| 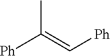 | 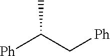 | 40% | 27%^{b} | 30 hr |
| 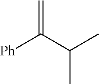 | 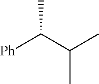 | 96% | 37%^{c,d} | 6 hr |
| 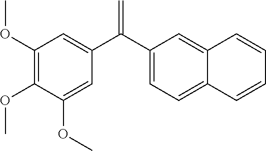 [SMILES: C=C(c1cc(OC)c(OC)c(OC)c1)c1ccc2ccccc2c1] | 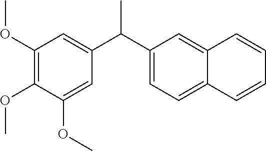 [SMILES: COc1cc(C(C)c2ccc3ccccc3c2)cc(OC)c1OC] | 15% | <5%^{b} | 16 hr |
| 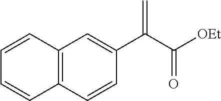 [SMILES: C=C(C(=O)OCC)c1ccc2ccccc2c1] | 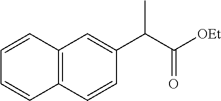 [SMILES: CCOC(=O)C(C)c1ccc2ccccc2c1] | 98%^{e} | <5%^{b} | 24 hr |
| 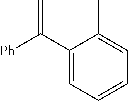 | 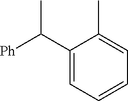 | 60% | <5%^{b} | 24 hr |

^{a}Conversions determined by GC-FID
^{b}% ee determined by chiral SFC-HPLC
^{c}% ee determined by chiral GC-FID
^{d}(R enantiomer)
^{e}Catalysis run at 0.1M [substrate]

### Example 9

### Hydrogenation of Three Ethylenically Unsaturated Compounds in the Presence of Compound E

Three ethylenically unsaturated compounds were hydrogenated with hydrogen in the presence of compound E, and the results are shown below in Table IV. In reaction vessels, 0.84M of the ethylenically unsaturated compound to be hydrogenated and 5 mol % of compound E were dissolved in 0.74 mL of benzene. Hydrogen (H₂) was introduced into the reaction vessels at a pressure of four atmospheres, and hydrogenation of this olefin was carried out at 25° C.

TABLE IV

| Ethylenically unsaturated | | | | |
|---|---|---|---|---|
| compound | Product | Conversion^{a} | % ee | time |
| 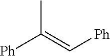 | 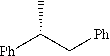 | 80% | 20%^{b} | 24 hr |
| 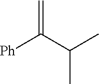 | 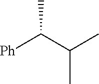 | 29% | 25%^{c,d} | 24 hr |
| 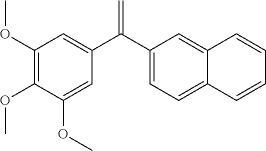 [SMILES: C=C(c1cc(OC)c(OC)c(OC)c1)c1ccc2ccccc2c1] | 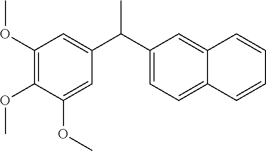 [SMILES: COc1cc(C(C)c2ccc3ccccc3c2)cc(OC)c1OC] | >98% | 7%^{b} | 36 hr |

^{a}Conversions determined by GC-FID
^{b}% ee determined by chiral SFC-HPLC
^{c}% ee determined by chiral GC-FID
^{d}(R enantiomer)
^{e}Catalysis run at 0.1M [substrate]

### Example 10

### Hydrogenation of methyl 2-acetamidoacrylate in the Presence of Compound D

Methyl 2-acetamidoacrylate (0.1 M) and compound D (5 mol %) were placed in a reaction vessel and dissolved in toluene at room temperature. Hydrogen (H₂) was introduced into the reaction vessel at a pressure of 500 psi. Compound D was the catalyst for this hydrogenation. The product obtained from this hydrogenation was methyl 2-acetamidoproponate. The conversion was 92% and the enantiomeric excess was 94%

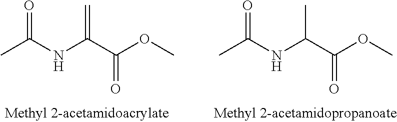

### Example 11

### Hydrogenation of methyl 2-acetamidoacrylate in the Presence of Compound E

The same experiment of Example 10 was carried out, except that compound D was replaced with compound E (the relative amounts of the compounds present were the same). The conversion of methyl 2-acetamidoacrylatemethyl to methyl 2-acetamidoproponate was 96% and the enantiomeric excess was 44%.

### Example 12

### Hydrogenations of E-α-methylstilbene in the Presence of Catalytic Compounds Generated In Situ

In separate experiments, E-α-methylstilbene (0.1 M), a ligand indicated in Table V, and (py)₂CoNs₂ (5 mol %) were placed in a reaction vessel and dissolved in toluene. Hydrogen (H₂) was introduced into the reaction vessel at a pressure of 500 psi, and hydrogenation reactions of E-α-methylstilbene were carried out for each ligand. It is believed that the catalytic compound is generated in situ. All of these hydrogenation reactions were carried out for 24 hours at room temperature. The conversion percentage and the enantiomeric excesses of E-α-methylstilbene to propane-1,2-diyldibenzene are reported for each experiment in Table V.

TABLE V

| | | | Enantiomeric |
|---|---|---|---|
| Example | Ligand | Conversion (%) | Excess (%) |
| 12-1 | SL-J851-2 | 86.1 | 28.6 |
| 12-2 | SL-A109-2 | 83.1 | 93.8 |
| 12-3 | SL-J034-1 | 80.1 | 62.2 |
| 12-4 | SL-J853-2 | 70.6 | 18.6 |
| 12-5 | SL-J408-1 | 67.5 | 15.7 |
| 12-6 | CarboPhos | 64.4 | 27.3 |
| 12-7 | SL-J418-1 | 41.5 | 14.7 |
| 12-8 | (S)-Binapine | 34.1 | 21.2 |
| 12-9 | (R)-DM-SegPhos | 27.4 | 58.4 |
| 12-10 | SL-J505-1 | 24.5 | 15.7 |
| 12-11 | SL-N004-2 | 24.1 | 43.0 |
| 12-12 | SL-A120-2 | 23.9 | 55.7 |
| 12-13 | (S,S)—Me-UCAP-DTBM | 23.8 | 16.7 |
| 12-14 | (R)-DTBM-SegPhos | 23.8 | 61.5 |
| 12-15 | SL-J417-1 | 22.3 | 71.6 |
| 12-16 | SL-J412-1 | 22.3 | 50.7 |
| 12-17 | (Rc,Sp)-DuanPhos | 22.0 | 43.1 |
| 12-18 | SL-J404-1 | 21.2 | 33.2 |
| 12-19 | SL-J220-1 | 17.0 | 47.3 |
| 12-20 | SL-J204-1 | 16.2 | 29.0 |
| 12-21 | SL-N007-2 | 15.4 | 49.2 |
| 12-22 | SL-J001-1 | 15.2 | 30.7 |
| 12-23 | SL-A102-1 | 14.7 | 49.2 |
| 12-24 | (S,S)-1,2-(MePPh)₂Ph | 13.9 | 31.9 |
| 12-25 | SL-J031-1 | 13.0 | 46.6 |
| 12-26 | (R,R)-BICP | 12.9 | 74.9 |

### Example 13

### Hydrogenation of E-α-methylstilbene in the Presence of Catalytic Compounds Generated In Situ

In a separate experiment, E-α-methylstilbene (0.04 M) was hydrogenated with hydrogen in the presence of a catalytic compound generated in situ using a different method. Cobalt dichloride (CoCl₂) was stirred with (R)-DTBM-SegPhos in tetrahydrofuran for 20 minutes. Upon filtration through celite and evaporation of solvent, the complex was dissolved in a 1.1 mL of tetrahydrofuran and 6.8 mL of toluene, and was placed in a metal high-pressure reaction vessel with E-α-methylstilbene (0.04 M), and 2 molar equivalents (relative to cobalt) of (trimethylsilyl)methyllithium. Hydrogen (H₂) was introduced into the metal high-pressure reaction vessel at a pressure of 500 psi, and the hydrogenation of E-α-methylstilbene was carried out for 1 hour at room temperature. It is believed that the catalyst was generated in situ. The conversion percentage of E-α-methylstilbene to propane-1,2-diyldibenzene was 33% and the enantiomeric excess was 84% favoring (S)-propane-1,2-diyldibenzene.

### Example 14

### Hydrogenation of E-α-methylstilbene in the Presence of Compound F

E-α-methylstilbene (0.84 M) and compound F (5 mol % [Co]) were placed in a reaction vessel and dissolved in 0.7 milliliters of benzene. Hydrogen (H₂) was introduced into the reaction vessel at a pressure of four atmospheres, and hydrogenation of this olefin was carried out for six hours at 25° C. Compound F was the catalyst for this hydrogenation. The product obtained from this hydrogenation was propane-1,2-diyldibenzene, and the conversion percentage of E-α-methylstilbene to propane-1,2-diyldibenzene was 4%. See Scheme 4, below:

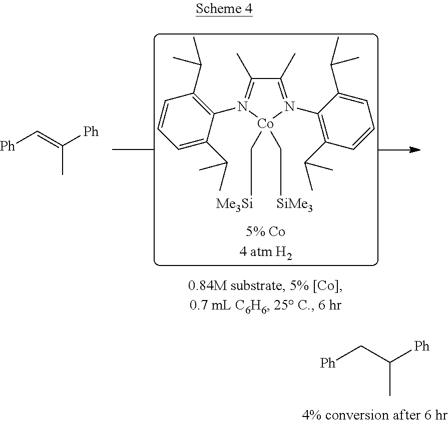

### Example 15

### Hydrogenation of (3-methylbut-1-en-2-yl)benzene in the presence of (K²-pybox)Co(Ns)₂

(3-methylbut-1-en-2-yl)benzene (0.86 M) and (κ²-pybox)Co(Ns)₂ (5 mol % [Co]), shown below in Scheme 4, were placed in a reaction vessel and dissolved in 650 mg of benzene. Hydrogen (H₂) was introduced into the reaction vessel at a pressure of four atmospheres, and hydrogenation of this olefin was carried out at 22° C. The compound (κ²-pybox)Co(Ns)₂ was the catalyst for this hydrogenation. The product obtained from this hydrogenation was (3-methylbutan-2-yl)benzene. The conversion percentage of (3-methylbut-1-en-2-yl)benzene to (3-methylbutan-2-yl)benzene was 50%. See Scheme 5, below:

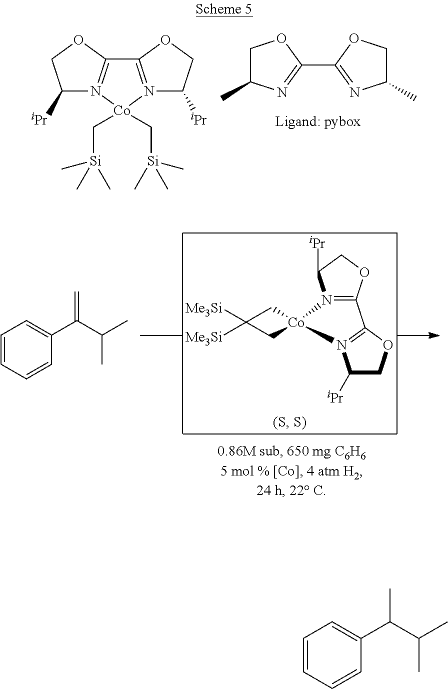

### Comparative Example 1

### Hydrogenation of E-α-methylstilbene in the Presence of Compound A and a Phosphine Additive

The same experiment of Example 2 was carried here except that 5 mol % of the additive triphenylphospine (PPh₃) was added to the reaction vessel. After 1 hour and 6 hours of reaction time, the conversion percentages were 41% and 48%, respectively. These data indicate that phosphine additives inhibit the hydrogenation of E-α-methylstilbene to propane-1,2-diyldibenzene. These results are consistent with the results disclosed in each of Hendrikse and Coenen (J. Catal. 1973, 30, 72-78) and Hendrikse et al. (Int. J. Chem. Kinet. 1975, 7, 557-574).

We claim:

1. A compound represented by formula (I): 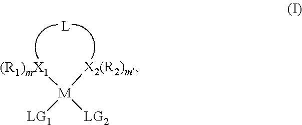
or a salt thereof,
wherein
M represents a metal atom selected from the group consisting of manganese, iron, cobalt, and nickel;
L represents linking group selected from the group consisting of a substituted or unsubstituted, straight-chain or branched, saturated or unsaturated C₁₋₄₀ hydrocarbyl group; a substituted or unsubstituted, saturated or unsaturated C₃₋₄₀ (hetero)cyclohydrocarbyl group; a substituted or unsubstituted C₃₋₄₀ (hetero)aryl group; and a metallocene where each aromatic ring has at least one substituted or unsubstituted, straight-chain or branched, saturated or unsaturated C₁₋₄₀ hydrocarbyl group that connects to one of X₁ and X₂;
each of X₁ and X₂, individually, represents an atom selected from the group consisting of nitrogen, phosphorus, arsenic, oxygen, sulfur, and selenium, with the proviso that X₁ represents nitrogen, phosphorus, or arsenic when X₂ represents oxygen, sulfur, or selenium, or with the proviso that X₁ represents oxygen, sulfur, or selenium when X₂ represents nitrogen, phosphorus, or arsenic;
each of LG₁ and LG₂, individually, represents a leaving group;
each R₁ and R₂, individually, represents a hydrogen atom, a substituted or unsubstituted, straight-chain or branched, saturated or unsaturated C₁₋₄₀ hydrocarbyl group; a substituted or unsubstituted, saturated or unsaturated C₃₋₄₀ (hetero)cyclohydrocarbyl group; and a substituted or unsubstituted C₃₋₄₀ (hetero)aryl group; or a halogen atom, where at least one hydrogen atom from at least one carbon atom in the L group is optionally removed to form a (hetero)cycle with at least one R₁ group and at least one R₂ group; and
each of m and m′, individually, represents 0 or 1 when X₁ or X₂ represents an atom selected from the group consisting of oxygen, sulfur, and selenium, or represents 1 or 2 when X₁ or X₂ represents an atom selected from the group consisting of nitrogen, phosphorus, and arsenic.

2. The compound according to claim 1, wherein M represents a manganese atom.

3. The compound according to claim 1, wherein M represents an iron atom.

4. The compound according to claim 1, wherein M represents a cobalt atom.

5. The compound according to claim 1, wherein M represents a nickel atom.

6. The compound according to claim 1, wherein each of X₁ and X₂ represents a nitrogen atom.

7. The compound according to claim 1, wherein each of X₁ and X₂ represents a phosphorus atom.

8. The compound according to claim 1, wherein X₁ represents a phosphorous atom and X₂ represents a nitrogen atom.

9. The compound according to claim 1, wherein X₁ represents a nitrogen atom and X₂ represents a phosphorus atom.

10. The compound according to claim 1, wherein X₁ represents a phosphorous atom and X₂ represents an oxygen atom.

11. The compound according to claim 1, wherein X₁ represents an oxygen atom and X₂ represents a phosphorus atom.

12. The compound according to claim 1, wherein M represents a cobalt atom, each of X₁ and X₂ represents a nitrogen atom, and L represents an ethylene group.

13. The compound according to claim 1, wherein M represents a cobalt atom; each of X₁ and X₂ represents a phosphorus atom, and L represents an ethylene group.

14. The compound according to claim 1, wherein M represents a cobalt atom, X₁ represents a phosphorus atom, X₂ represents a nitrogen atom, and L represents an ethylene group.

15. The compound according to claim 1, wherein M represents a cobalt atom, X₁ represents a nitrogen atom, X₂ represents a phosphorus atom, and L represents an ethylene group.

16. The compound according to claim 1, wherein M represents a cobalt atom, X₁ represents a phosphorus atom, X₂ represents an oxygen atom, and L represents an ethylene group.

17. The compound according to claim 1, wherein M represents a cobalt atom, X₁ represents an oxygen atom, X₂ represents a phosphorus atom, and L represents an ethylene group.

18. The compound according to claim 1, wherein M represents a cobalt atom, each of X₁ and X₂ represents a phosphorus atom, L represents an ethylene group, and each of Y₁ and Y₂ represents a silicon atom.

19. The compound according to claim 1, wherein each R₂ and R₃ group is an ethyl group.

20. The compound according to claim 1, wherein each R₂ and R₃ group is a phenyl group.

21. The compound according to claim 1, wherein R₁)ₘX₁-L-X₂(R₂)_{m′} represents (S)-(6,6′-dimethoxybiphenyl-2,2′-diyl)bis[bis(3,5-di-tert-butyl-4-methoxyphenyl)phosphine.

22. The compound according to claim 1, wherein (R₁)ₘX₁-L-X₂(R₂)_{m′} represents a bidentate ligand selected from the group consisting of dppe, depe, chiraphos, duphos, and duanphos.

23. The compound according to claim 1, wherein (R₁)ₘX₁-L-X₂(R₂)_{m′} represents a bidentate ligand selected from the group consisting of dppe, depe, chiraphos, and duanphos.

24. The compound according to claim 1, which is

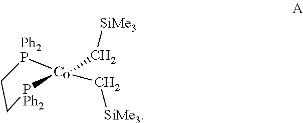

25. The compound according to claim 1, which is

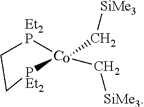

26. The compound according to claim 1, which is

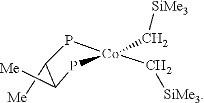

27. The compound according to claim 1, which is

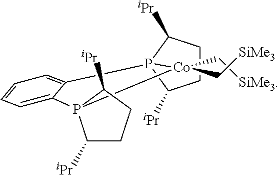

28. The compound according to claim 1, which is

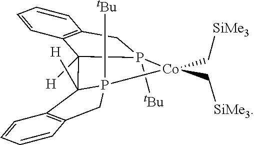

29. The compound according to claim 1, which is 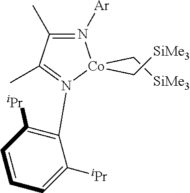
wherein Ar represents a 2,6-diisopropylphenyl group.

30. A method of making a compound according to claim 1, comprising reacting (py)₂M(LG₁)(LG₂) with (R₁)ₘX₁-L-X₂(R₂)_{m′} to form the compound, where py represents pyridine.

31. A method of making a compound according to claim 1, comprising reacting M(LG₁)(LG₂) with (R₁)ₘX₁-L-X₂(R₂)_{m′}, followed by treatment with a reducing agent to form the compound.

32. The method according to 30, wherein said reacting is carried out in a solvent comprising diethyl ether and at a temperature of from −60° to 40° C.

33. A catalyst comprising at least one compound according to claim 1.

34. A method, comprising hydrogenating an olefin in the presence of a catalyst according to claim 33.
